(19) United States
(12) Patent Application Publication
(10) Pub. No.: US 2026/0305739 A1
(43) Pub. Date: Oct. 8, 2026

(54) Wearable Compression Apparatus for Localized Therapeutic Treatment

(71) Applicant: Angela Rosendale, Ettrick, WI (US)

(72) Inventor: Angela Rosendale, Ettrick, WI (US)

(21) Appl. No.: 19/461,218

(22) Filed: Jan. 27, 2026

Related U.S. Application Data
(60) Provisional application No. 63/769,322, filed on Mar. 10, 2025.

(51) Int. Cl. A41D 13/005; A41D 13/12; A41D 27/20; A61F 7/00; A61F 7/02; A61N 5/06

(52) U.S. Cl. CPC A41D 13/0058; A41D 13/0051; A41D 13/0053; A41D 13/1254; A41D 27/201; A61N 5/0625; A41D 2300/32; A41D 2300/322; A41D 2300/326; A41D 2500/50; A61F 2007/0041; A61F 2007/0042; A61F 2007/0043; A61F 2007/0044; A61F 2007/0096; A61F 2007/0238; A61N 2005/0645; A61N 2005/0659

(57) ABSTRACT

The present invention is a therapeutic garment that integrates cryotherapy and thermotherapy functionality into a wearable, form-fitting garment resembling athletic wear. The garment includes a waistband and leg-enclosing members made from flexible, breathable, and compressive materials to provide support during movement. A plurality of therapeutic pockets are strategically positioned on the waistband and along the legs to target muscle groups and joints such as the abdomen, ankle, quads, hamstrings, calves, knees, glutes, and lumbar region. Each pocket includes an opening for receiving a cryotherapy or thermotherapy pack and is secured by a closure. In some embodiments, pockets feature removable liners to reduce moisture buildup and integrated flexible temperature sensors to monitor pack temperature. The garment may include adjustable belts and compression straps to ensure fit and enhance abdominal compression.

### CROSS-REFERENCE TO RELATED APPLICATION

The present application claims priority to, and the benefit of, U.S. Provisional Application No. 63/769,322, which was filed on Mar. 10, 2025, and is incorporated herein by reference in its entirety.

### FIELD OF THE INVENTION

The present invention generally relates to wearable therapeutic garments. More specifically, the invention relates to a form-fitting activewear garment integrated with strategically placed pockets for cryotherapy and thermotherapy. The garment includes a plurality of integrated pockets and enables users to insert reusable hot or cold packs into designated pockets for providing localized thermal treatment. The garment provides the appearance and flexibility of traditional athletic wear while delivering localized thermal treatment. The garment is constructed to contour the body and provide gentle compression. Accordingly, the present disclosure makes specific reference thereto. Nonetheless, it is to be appreciated that aspects of the present invention are also equally applicable to other like applications, devices, and methods of manufacture.

### BACKGROUND

By way of background, individuals commonly use applications of therapeutic heat or cold, such as heating packs or ice packs for managing pain, inflammation, fat reduction, and muscle recovery following exercise, physical strain, injury, or surgical procedures. However, traditional methods of applying such therapy can be inconvenient and ineffective. Users often must hold the pack manually against the affected area, which limits mobility and leaves only one hand available for other tasks. Moreover, improperly positioned packs may shift or fall out of place, reducing the efficacy of the treatment and requiring constant adjustment. Use of conventional packs is difficult during travel and on-the-go.

Also, for toning the body, individuals seek non-invasive methods, particularly in regions with excess white adipose tissue. Cryotherapy has been shown to assist in reducing white fat cells, yet there remains a lack of easy-to-use solutions, such as wearable solutions that can deliver targeted cooling therapy effectively and discreetly. Accordingly, individuals desire an improved solution that offers secure, mobile, and targeted delivery of hot or cold therapy to key areas of the body in an easy-to-use and non-invasive way.

Therefore, there exists a long-felt need in the art for a therapeutic garment that enables users to apply hot or cold therapy to specific areas of the body without needing to manually hold a therapy pack in place. There is a long-felt need for a wearable solution that supports mobility and enables users to carry out daily tasks while receiving therapeutic treatment. Additionally, there exists a long-felt need for a garment that ensures the consistent positioning of thermal packs over muscles and joints without slipping or discomfort. Furthermore, there is a long-felt need for therapeutic garments that offer a secure, compressive, and aesthetically appealing alternative to traditional wraps or handheld packs. More specifically, there is a long-felt need for garments that can aid in targeted cryotherapy for reducing white fat cells and for accelerating muscle recovery. Finally, there is a long-felt need for an activewear-style garment that integrates flexible therapy pockets and supports use during both rest and motion.

The subject matter disclosed and claimed herein, in one embodiment, comprises a therapeutic garment in the form of athletic leggings or compression pants. The garment includes a high waistband and a pair of leg-enclosing members made from flexible, breathable, and durable materials such as spandex, polyester, or performance fabric/material blends. The garment is constructed to contour the body and provide gentle compression. A plurality of strategically placed therapeutic pockets are integrated into the waistband and the pair of leg-enclosing members and are configured to hold reusable cryotherapy or thermotherapy packs. The pockets are positioned over, or adjacent to, key recovery zones, including the abdomen, thighs, knees, calves, and hamstrings. Each pocket includes an opening secured by a closure to hold the therapy pack in place securely during activity or rest. The therapeutic garment enables hands-free therapy during motion or rest without disrupting the user's daily routine.

In another embodiment, the therapeutic pockets may include features such as removable liners to reduce moisture buildup from condensation and flexible temperature sensors to monitor the temperature of the therapeutic packs.

In this manner, the therapeutic garment of the present invention fulfills the long-felt need for a hands-free, mobile, and form-fitting wearable system for administering localized heat or cold therapy. The invention supports physical rehabilitation, muscle recovery, and aesthetic toning through cryo-induced fat reduction, all while being discreet and wearable as everyday activewear. The garment enables users to recover or manage pain while remaining productive, mobile, or at rest without compromising therapeutic efficacy or comfort. The integrated pockets and removable liners make the garment a versatile solution for athletes, post-surgery patients, fitness enthusiasts, and others seeking targeted recovery and body care.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a therapeutic garment. The therapeutic garment comprises a waist cover portion, a pair of leg-enclosing members extend downward from the waist cover portion, a plurality of integrated therapeutic pockets are disposed on the waist cover portion and the leg-enclosing members. Each therapeutic pocket comprises an opening configured to receive a cryotherapy or thermotherapy pack and a closure mechanism for securing the cryotherapy or thermotherapy pack within the pocket, wherein the therapeutic garment is constructed from a flexible, breathable, and compressive material and is configured to contour a user's body while enabling mobility during therapy.

In another aspect, a therapeutic garment is disclosed. The therapeutic garment includes a garment body including a waistband and leg portions extending to an ankle end, a set of therapeutic pockets positioned over target muscle or joint zones including at least one of: abdomen, ankle, quads, hamstrings, calves, knees, glutes, or lumbar region, each therapeutic pocket has an opening aligned in a vertical or horizontal orientation and is configured to receive a removable hot or cold therapeutic pack.

In another embodiment, the waistband includes a plurality of adjustable belts, each belt is coupled to a corresponding clip to secure the garment to users of different waist sizes, a compressive strap is integrated into the waistband and is configured to be pulled to tighten the waistband around the abdomen of a wearer, and a plurality of therapeutic pockets are integrated into the waistband, each pocket is configured to retain a cryotherapy or thermotherapy pack.

In one embodiment, the pocket includes a removable inner liner configured to reduce moisture build-up from condensation, and a flexible temperature sensor is embedded in the pocket. The sensor is configured to monitor the temperature of the therapeutic pack and indicate when the temperature exceeds a predetermined safety threshold.

In yet another aspect, the zipper has a longitudinal orientation and is configured to enable partial opening to facilitate ventilation or quick removal of the cryotherapy or thermotherapy pack.

In a further embodiment, the therapeutic packs are interchangeable and include different shapes selected from the group consisting of curved packs for knees, flat packs for thighs, and wedge-shaped packs for ankles.

In still another embodiment, the pocket is further configured to receive at least one of an electro muscle stimulation (EMS) pad or an infrared heating pad in addition to or in place of the therapeutic pack.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of the therapeutic garment of the present invention in accordance with the disclosed structure;

FIG. 2 illustrates a rear perspective view of the therapeutic garment 100 of the present invention in accordance with the disclosed structure;

FIG. 3 illustrates an enlarged view of the waistband and plurality of pockets integrated thereto in accordance with the disclosed structure;

FIG. 4 illustrates a perspective view showing a therapeutic pack being added to a pocket for use in accordance with the disclosed structure; and

FIG. 5 illustrates an isolated view of another embodiment of the pocket integrated into the therapeutic garment of the present invention in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a therapeutic garment that enables users to apply hot or cold therapy to specific areas of the body without needing to manually hold a therapy pack in place. There is a long-felt need for a wearable solution that supports mobility and enables users to carry out daily tasks while receiving therapeutic treatment. Additionally, there exists a long-felt need for a garment that ensures the consistent positioning of thermal packs over muscles and joints without slipping or discomfort. Furthermore, there is a long-felt need for therapeutic garments that offer a secure, compressive, and aesthetically appealing alternative to traditional wraps or handheld packs. More specifically, there is a long-felt need for garments that can aid in targeted cryotherapy for reducing white fat cells and for accelerating muscle recovery. Finally, there is a long-felt need for an activewear-style garment that integrates flexible therapy pockets and supports use during both rest and motion.

The present invention, in one exemplary embodiment, is a therapeutic garment. The therapeutic garment includes a garment body including a waistband and leg portions extending to an ankle end, a set of therapeutic pockets positioned over target muscle or joint zones including at least one of: abdomen, ankle, quads, hamstrings, calves, knees, glutes, or lumbar region, each therapeutic pocket has an opening aligned in a vertical or horizontal orientation and is configured to receive a removable hot or cold therapeutic pack. By integrating therapy directly into the therapeutic garment, the therapeutic garment fulfills the long-standing need for a mobile, customizable, and wearable therapeutic solution and enables individuals to go about their day while receiving effective, targeted cryotherapy and/or thermotherapy.

Reference will now be made in detail to the present preferred embodiments of the invention, examples of which are illustrated in the accompanying drawings. Wherever possible, the same reference numerals are used in the drawings and the description to refer to the same or like parts.

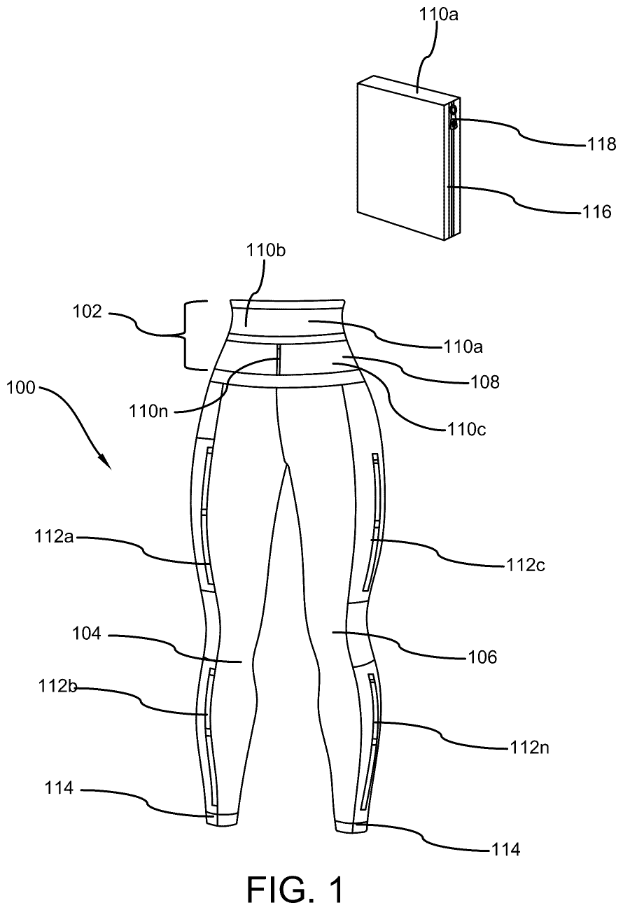

Referring initially to the drawings, FIG. 1 illustrates a front perspective view of the therapeutic garment of the present invention in accordance with the disclosed structure. The therapeutic garment 100 of the present invention is designed to combine the utility of cryotherapy (cold therapy) and thermotherapy (heat therapy) with the convenience, flexibility, and look of sportswear. More specifically, the therapeutic garment 100 enhances recovery, provides localized pain relief, and supports metabolic fat reduction through regular use. The therapeutic garment 100 has the appearance of athletic pants or leggings and can be worn as everyday activewear.

The therapeutic garment 100 includes a waist cover portion 102 in the form of a high waistband and a pair of leg enclosing members 104, 106. The therapeutic garment 100 is made from flexible, breathable, and durable fabric/materials like spandex, nylon, polyester, or performance-grade blends. Furthermore, the therapeutic garment 100 has a form-fitting construction and contours the body for support and compression.

The therapeutic garment 100 includes an integrated therapeutic pockets system 108, which includes a first set of strategically placed pockets 110a-n on the waistband 102 and a second set of pockets 112a-n running from the waist to the ankle end 114 of each leg enclosing members 104, 106. It should be noted that each pocket of the sets of pockets is designed to be positioned over, or adjacent to, key muscle groups or joint areas such as quads, hamstrings, calves, knees, glutes, and lumbar region. Each pocket (shown exemplary through 110a) of the therapeutic garment 100 has a built-in opening 116 for sliding and holding an ice pack or a heat pack. Further, the built-in opening 116 of each pocket is secured using a closure 118 such as zippers, hook-and-loop fasteners, or buttons.

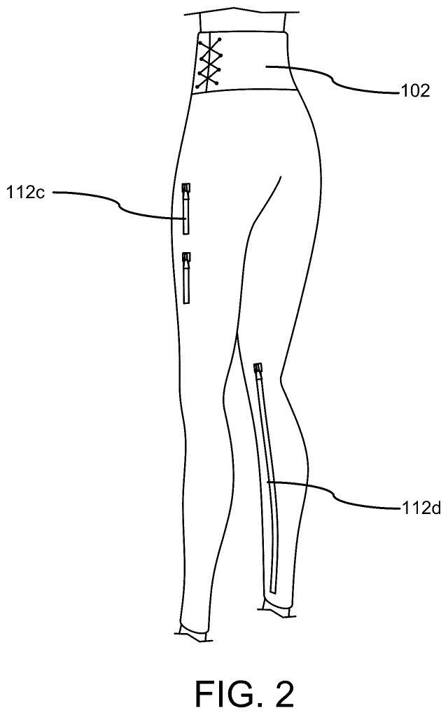

FIG. 2 illustrates a rear perspective view of the therapeutic garment 100 of the present invention in accordance with the disclosed structure. The therapeutic garment 100 is used for providing therapeutic effects on calves and knees as well. As illustrated, the pocket 112d helps reduce inflammation, pain, and swelling while helping the garment 100 to contour around the leg of a user. The waistband 102 compresses the abdomen of the wearer and helps in providing effective therapeutic effect.

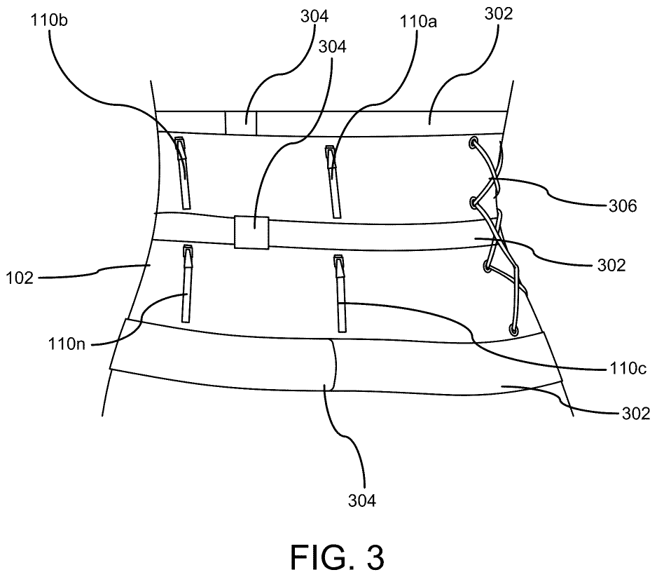

FIG. 3 illustrates an enlarged view of the waistband and plurality of pockets integrated thereto in accordance with the disclosed structure. The waistband 102 includes a plurality of belts 302 for securing the therapeutic garment 100 to different users. Each belt of the plurality of belts 302 is adjustable using a corresponding clip 304 for adjusting the length of the belt. Each pocket of the plurality of pockets 110a-n is configured to hold an ice pack or a heat pack for providing a therapeutic effect. For further adjusting the waistband 102 and compressing the abdomen of a user, a strap 306 is integrated into the waistband 102, wherein the strap 306 can be pulled or cinched to further squeeze or reduce (i.e., compress) the waistband 102 around the user.

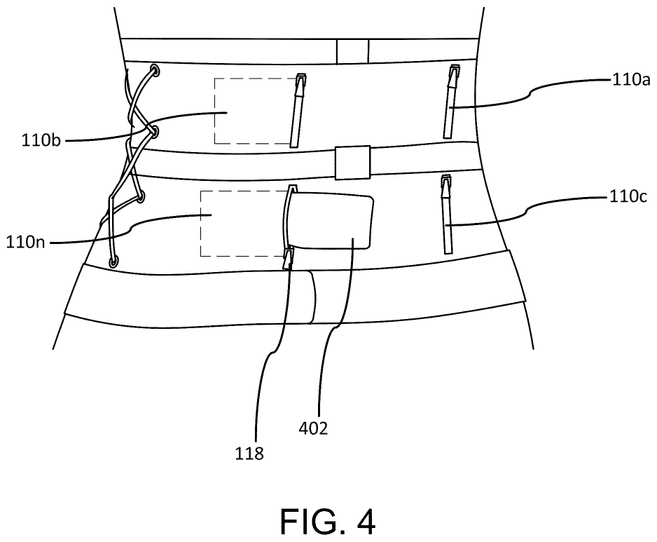

FIG. 4 illustrates a perspective view showing a therapeutic pack being added to a pocket for use in accordance with the disclosed structure. One advantage of the therapeutic garment 100 (also referred to as cryo-thermic active wear) is that it enables mobility during use of therapeutic packs. A therapeutic pack 402, which is preferably a glycol-based hot or cold gel pack, has dimensions of about 15″×11″ and is reusable. The pack can be either cooled in the freezer or heated in a microwave, depending on the desired therapy. The therapeutic pack 402 slides into a pocket (such as 110n) through the opening 116, and the pocket is stretched to conform to the shape and size of the therapeutic pack 402. Once the therapeutic pack 402 is inside the pocket, the zipper 118 is fastened, and the therapeutic pack 402 remains snug and flat against the body of the wearer. It should be noted that the therapeutic pack 402 can come in different sizes and shapes, and further, the opening of each pocket can be aligned vertically or horizontally, depending on the position of the pocket.

In some embodiments, different shapes and sizes of cryo/thermo packs can be used for different zones, such as curved for knees, flat for thighs, and wedge-shaped for ankles. The pockets are also compatible with EMS pads (electro muscle stimulation) and infrared heating pads for providing effective therapeutic effects.

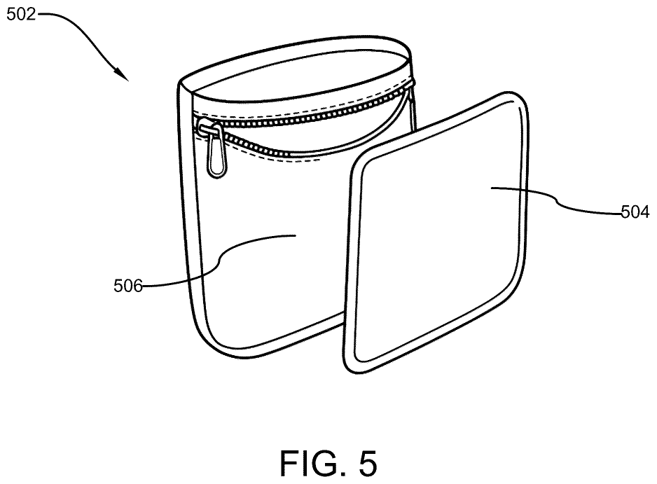

FIG. 5 illustrates an isolated view of another embodiment of the pocket integrated into the therapeutic garment of the present invention in accordance with the disclosed structure. As illustrated, the pocket 502 in the present embodiment includes a removable liner 504 to prevent moisture build-up from condensation. Further, the pocket 502 has a flexible temperature sensor 506 to prevent overheating or freezing and may indicate when the temperature is not safe for human skin.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein, “therapeutic garment”, “cryo-thermic active wear”, “form-fitting activewear garment”, and “garment” are interchangeable and refer to the therapeutic garment with integrated cryotherapy and thermotherapy pocket system 100 of the present invention.

Notwithstanding the foregoing, the therapeutic garment with integrated cryotherapy and thermotherapy pocket system 100 of the present invention can be of any suitable configuration as is known in the art without affecting the overall concept of the invention, provided that it accomplishes the above-stated objectives. One of ordinary skill in the art will appreciate that the therapeutic garment with integrated cryotherapy and thermotherapy pocket system 100 as shown in the FIGS. are for illustrative purposes only, and that many other configurations of the therapeutic garment with integrated cryotherapy and thermotherapy pocket system 100 are well within the scope of the present disclosure. Although the dimensions of the therapeutic garment with integrated cryotherapy and thermotherapy pocket system 100 are important design parameters for user convenience, the therapeutic garment with integrated cryotherapy and thermotherapy pocket system 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising,” as “comprising” is interpreted when employed as a transitional word in a claim.

What is claimed is:

1. A therapeutic garment comprising:
a wearable therapeutic garment having a waist cover portion, a waistband, and a pair of leg enclosing members;
wherein said waist cover portion having a first set of pockets;
wherein a first leg of said pair of leg enclosing members having a second set of pockets;
wherein a second leg of said pair of leg enclosing members having a third set of pockets;
wherein said first set of pockets having at least one pocket adjacent to a lumbar region;
wherein said second set of pockets having a plurality of pockets adjacent to areas selected from the group consisting of a quad, a hamstring, a calve, a knee, an ankle, and a glute;
wherein said third set of pockets having another plurality of pockets adjacent to areas selected from the group consisting of a quad, a hamstring, a calve, a knee, an ankle, and a glute; and
further wherein each one of said first set of pockets, said second set of pockets, and said third set of pockets having a built-in opening for selectively receiving a therapeutic pack.

2. The therapeutic garment of claim 1, wherein said therapeutic pack selected from the group consisting of a cryotherapy pack, a thermotherapy pack, an EMS pad, and an infrared heating pad.

3. The therapeutic garment of claim 2, wherein each of said built-in opening having a selectable closure selected from the group consisting of a zipper, a hook-and-loop fastener, and a button.

4. The therapeutic garment of claim 3, wherein said wearable therapeutic garment has a form-fitting construction and contours the body for support and compression.

5. The therapeutic garment of claim 4, wherein said wearable garment having a material selected from the group consisting of a spandex, a nylon, and a polyester.

6. The therapeutic garment of claim 5, wherein said waistband having a plurality of belts for securing said wearable therapeutic garment to a user.

7. The therapeutic garment of claim 6, wherein each belt of said plurality of belts having a clip for adjusting a length of said each belt.

8. The therapeutic garment of claim 7, wherein said waistband having an adjustable strap for cinching said waist cover portion around a waist of the user.

9. A therapeutic garment comprising:
a wearable therapeutic garment having a waist cover portion, a waistband, and a pair of leg enclosing members;
wherein a first leg of said pair of leg enclosing members having a first set of pockets;
wherein a second leg of said pair of leg enclosing members having a second set of pockets;
wherein said first set of pockets having a plurality of pockets adjacent to areas selected from the group consisting of a quad, a hamstring, a calve, a knee, an ankle, and a glute;
wherein said second set of pockets having another plurality of pockets adjacent to areas selected from the group consisting of a quad, a hamstring, a calve, a knee, an ankle, and a glute;
wherein each one of said first set of pockets and said second set of pockets having a built-in opening for selectively receiving a therapeutic pack; and
further wherein said therapeutic pack selected from the group consisting of a cryotherapy pack, a thermotherapy pack, an EMS pad, and an infrared heating pad.

10. The therapeutic garment of claim 9, wherein said waist cover portion having a third set of pockets, and further wherein said third set of pockets having at least one pocket adjacent to a lumbar region.

11. The therapeutic garment of claim 10, wherein each of said built-in opening having a selectable closure selected from the group consisting of a zipper, a hook-and-loop fastener, and a button.

12. The therapeutic garment of claim 11, wherein said wearable therapeutic garment has a form-fitting construction and contours the body for support and compression.

13. The therapeutic garment of claim 12, wherein said wearable garment having a material selected from the group consisting of a spandex, a nylon, and a polyester.

14. The therapeutic garment of claim 13, wherein said waistband having a plurality of belts for securing said wearable therapeutic garment to a user.

15. The therapeutic garment of claim 14, wherein each belt of said plurality of belts having a clip for adjusting a length of said each belt.

16. The therapeutic garment of claim 15, wherein said waistband having an adjustable strap for cinching said waist cover portion around a waist of the user.

17. A therapeutic garment comprising:
a wearable therapeutic garment having a waist cover portion, a waistband, and a pair of leg enclosing members;
wherein a first leg of said pair of leg enclosing members having a first set of pockets;
wherein a second leg of said pair of leg enclosing members having a second set of pockets;
wherein said first set of pockets having a plurality of pockets adjacent to areas selected from the group consisting of a quad, a hamstring, a calve, a knee, an ankle, and a glute;
wherein said second set of pockets having another plurality of pockets adjacent to areas selected from the group consisting of a quad, a hamstring, a calve, a knee, an ankle, and a glute;
wherein each one of said first set of pockets and said second set of pockets having a built-in opening for selectively receiving a therapeutic pack;
wherein said therapeutic pack selected from the group consisting of a cryotherapy pack, a thermotherapy pack, an EMS pad, and an infrared heating pad;
wherein said waist cover portion having a third set of pockets;
wherein said third set of pockets having at least one pocket adjacent to a lumbar region; and
further wherein said each one of said first set of pockets, said second set of pockets, and said third set of pockets having a flexible temperature sensor.

18. The therapeutic garment of claim 17, wherein said waistband having a plurality of belts for securing said wearable therapeutic garment to a user.

19. The therapeutic garment of claim 18, wherein each belt of said plurality of belts having a clip for adjusting a length of said each belt.

20. The therapeutic garment of claim 19, wherein said waistband having an adjustable strap for cinching said waist cover portion around a waist of the user.
